(19) United States
(12) Patent Application Publication
(10) Pub. No.: US 2008/0295830 A1
(43) Pub. Date: Dec. 4, 2008

(54) Tool and method for customized inhalation

(75) Inventors: Teddy Blayne Martonen, Laguna Beach, CA (US); Gabriela Apiou, Paris (FR); Marine Pichelin, Draveil (FR); Ira Katz, Paris (FR); Joelle Texereau, Paris (FR); Georges Caillibotte, Allauch (FR)

Correspondence Address: YOUNG & THOMPSON, 209 Madison Street, Suite 500, ALEXANDRIA, VA, 22314, US

(73) Assignee: L'AIR LIQUIDE, SOCIETE ANONYME POUR L'ETUDE ET L'EXPLOITATION DES PROCEDES GEORGES CLAUDE, Paris (FR)

(21) Appl. No.: 11/806,136

(22) Filed: May 30, 2007

(51) Int. Cl. A61M 16/10

(52) U.S. Cl. 128/203.15

(57) ABSTRACT

A method for designing and/or customizing the treatment or for treating by inhalation of a patient suffering from a respiratory or non-respiratory disease including the steps of presenting a patient suffering from the disease; determining the kind of disease of the patient; prescribing at least one drug and/or gas and at least one kind of device to be used for administrating the drug or gas; inputting into a processing element, data chosen among the drug and/or prescribed gas and drug particle size obtained with the prescribed device; inputting into the processing element at least one patient characteristics of the patient; inputting into processing element at least one morphology and/or ventilatory condition of the patient; running a code in the processing element using the input data and conditions thereby calculating a flow and/or deposition characteristics customized to the patient.

The present invention concerns a method and a device for calculating flow and particle deposition characteristics of gas and/or one or several given inhaled pharmacologic drugs or airborne toxicants in human and animal airways and for providing a customized morphology-based and ventilation-based design of a treatment or exposure protocol. In the preceding statement, the reference to human subjects applies to medical and toxicological applications whereas for animals the application is for surrogate inhalation tests.

Using inhalable drugs and/or gas for treating some kinds of diseases is known. However, a remaining problem to be solved is to be able to inhale gas and/or pharmacologic drugs in such a way that they are effectively and efficiently targeted to appropriate regions of the respiratory system of the patient to treat diseases. This is the case whether the diseases to be treated are diseases of the respiratory system, e.g., asthma, or diseases such as diabetes, in which the lung is used as an avenue of entrance into the body for the systemic delivery of insulin.

In other words, inhaling gas and/or drugs can be an efficient treatment only if the drug particles reach the most desired regions of the respiratory system, e.g. the extra thoracic passages (ET). or the lungs and their tracheobronchial (TB) or pulmonary (P) compartments.

Providing an efficient method and device for evaluating the appropriate deposition areas in the contiguous respiratory system of a given patient is however not obvious at all conceptually and it is not straightforward technically using current state of the art protocols.

Indeed, if many documents present particle deposition models for the human respiratory system, they all disclose inconsistent mathematical systems of equations obtained by illegitimately coupling the Landahl, Beckmans and Ingham equations as described below, which lead to bad or insufficient deposition targeting.

Only Martonen, one of the inventors of the present invention, has used his own equations in his peer reviewed publications in which he was either sole author or co-author.

A goal of the present invention is hence to propose efficient method and device able to calculate flow and/or deposition characteristics of gas and/or one or several given inhaled pharmacologic drugs in human airways and to provide to the physician, health-care professionals, (e.g., nurses, respiratory technicians), a customized morphology-based and ventilation-based design of the treatment for a given patient

A solution of the invention is a method for designing and/or customizing the treatment or a method of treatment by inhalation of a patient suffering from a respiratory or non-respiratory disease comprising the steps of:
a) presenting a patient suffering from said disease,
b) determining the kind of disease of said patient,
c) prescribing at least one drug and/or gas and at least one kind of device to be used for administrating said drug or gas, based on the determination done in step b),
d) inputting into processing means, data chosen among said drug and/or gas prescribed in step c) and drug particle size obtained with the device prescribed in step c),
e) inputting into the processing means at least one patient characteristics, such as gender, age, height, race . . . etc of the said patient
f) inputting into processing means at least one morphology and/or ventilatory condition of said patient,
g) running a code in said processing means using the input data and conditions of step d), e) and f), thereby calculating a flow and/or deposition characteristics customized to said patient.

Further, the method of the present invention can comprise one or several of the following features:
it comprises a step of targeting the pulmonary region (P) to enhance drug deposition in alveolated airways for uptake by blood for delivery to non-lung sites, in the case where the disease is a non-respiratory disease.
it comprises a step of targeting sites in the tracheobronchial tree (TB) and/or in the pulmonary region (P) to enhance drug deposition in the airways, in the case where the disease is a respiratory disease.
the determination of the kind of disease in step b) is done by a physician.
in step b), the kind of respiratory disease is chosen among asthma, emphysema, chronic obstructive pulmonary disease (COPD), cystic fibrosis and cancer.
in step b), the kind of non-respiratory disease is chosen among diabetes, neurological disorders, influenza.
in step c), the gas is chosen among air and/or mixtures and drug is chosen among broncho-dilatators, steroids, antibiotics, pain killers, insulin, proteins, chemotherapies or gene therapies.
in step c), the device is an metered dose inhaler (MDI), a dry powder inhaler (DPI) or a nebulizer.
the processing means comprise a computer having at least one microprocessor for processing data, in particular for performing calculations and/or obtaining information and/or storing data and/or displaying results.
the customized flow and/or deposition characteristics calculated in step g) are displayed electronically on a screen, in particular a computer monitor or similar.
the input of data of steps d), e) and/or f) is operated by means of a graphic user interface (GUI), and access to the GUI is accomplished via a keyboard and/or a mouse.
in step g), the code comprises algorithms translating algebraic formulas describing the physics of aerosol and fluid motion in human respiratory systems for various breathing conditions.
customized flow and deposition characteristics calculated in step g) in various formats are displayed comprising total deposition in lungs, compartmental deposition within the TB tree and P region, localized deposition on a generation by generation basis, and/or mass (micrograms)-deposition on a per unit surface area basis, pressure drops on a generation by generation basis, mean velocity on a generation by generation basis.

The invention also concerns a medical device for designing and/or customizing the treatment by inhalation of a patient suffering from a respiratory or non-respiratory disease comprising:
input/output means interactive with the processing means:
   for inputting into processing means:
      data chosen among a prescribed drug and/or gas and a drug particle size obtained with a prescribed inhalation device,
      at least one patient characteristics such as gender, age, height, race and
      at least one morphology and/or ventilatory condition of said patient,
   and for outputting customized flow and/or deposition characteristics,
storing means for storing a code comprising a digital memory that is adapted to store algorithms translating algebraic formulas describing the physics of aerosol kinetics and fluid motion in human respiratory systems for various ventilatory conditions,
processing means cooperating with the storing means and the input means for running the code stored by the storing means and using the input data, and
displaying means for displaying customized flow and/or deposition characteristics.

The displaying means is a screen, in particular a computer monitor or similar.

Besides, the storing means for storing the code are chosen among CD-Rom, DVD-Rom, USB sticks and computer hard drives.

According to another aspect, the invention also deals with a method for designing the exposure by inhalation of a laboratory test animal used as a human surrogate in pharmacological and/or toxicological experiments, comprising the steps of:
h) presenting a selected laboratory test animal that is chosen to mimic a given human disease,
i) choosing at least one drug and/or gas to be inhaled by the test animal based on the selection done in step i),
j) choosing a device to be used for the inhalation by the test animal of the drug and/or gas chosen in step i),
k) determine operating conditions for the device chosen in step j) so that gas and/or particles of chosen sizes are produced so as to selectively deposit in the test animal,
l) inputting into processing means, data chosen among said gas and/or drug to be tested chosen in step i) and the drug particle size determined in step k),
m) inputting the morphology of the test animal,
n) inputting into processing means at least one ventilatory condition of said test animal,
o) running a code in said processing means using the input data and condition of step l), m) and n), thereby calculating a flow and/or deposition characteristics customized to said test animal,

Preferably, the test animal is chosen among rats, mice, guinea pigs, hamsters, dogs, donkeys, cats, swine and monkeys.

According to still another aspect, the invention concerns a method for designing the exposure by inhalation a human volunteer in toxicological experiments, comprising the steps of:
p) presenting a human volunteer,
q) choosing at least one substance to be inhaled by the human volunteer,
r) choosing a device to be used for the inhalation by the human volunteer of the substance chosen in step q),
s) determine operating conditions for the device chosen in step r) so that gas and/or particles of chosen sizes are selectively deposit in the human volunteer,
t) inputting into processing means, data chosen among said substance to be tested chosen in step t) and the substance particle size determined in step s),
u) inputting into the processing means at least one human volunteer characteristics such as gender, age, height, race
v) inputting into processing means at least one morphology and/or ventilatory condition of said human volunteer,
w) running a code in said processing means using the input data and condition of step t), u) and v), thereby calculating a flow and/or deposition characteristics customized to said human volunteer.

The present invention will be explained more in details thanks to the description below and accompanying drawings.

The following description and the enclosed figures show the effects of physical activity on deposition patterns of inhaled particles, where:

FIG. 1 represents lung morphology from MRI,

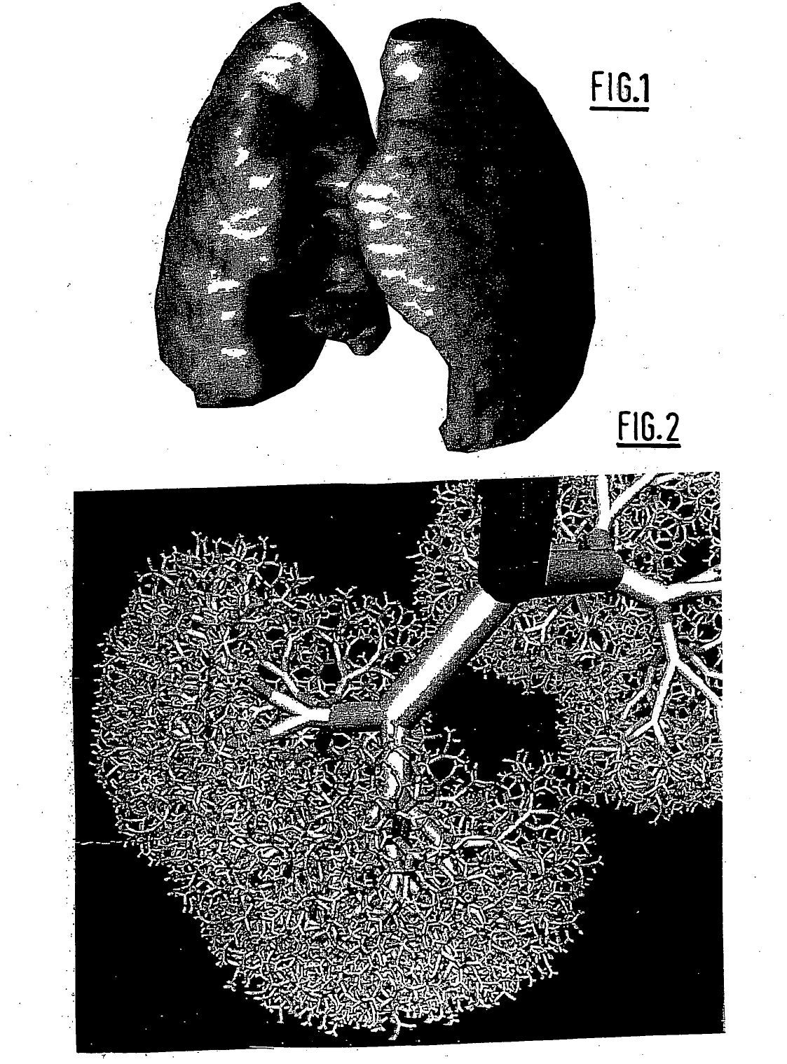

FIG. 2 represents the airway network in a lung,

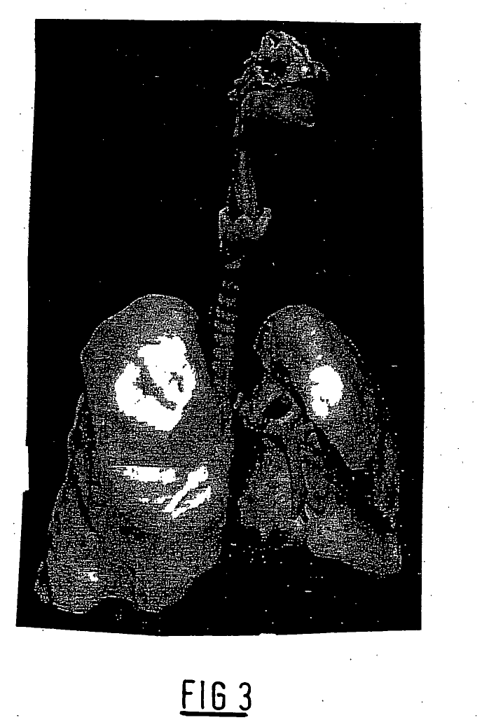

FIG. 3 represents a three-dimensional view of the contiguous respiratory system,

FIG. 4 represents flow patterns in the nose,

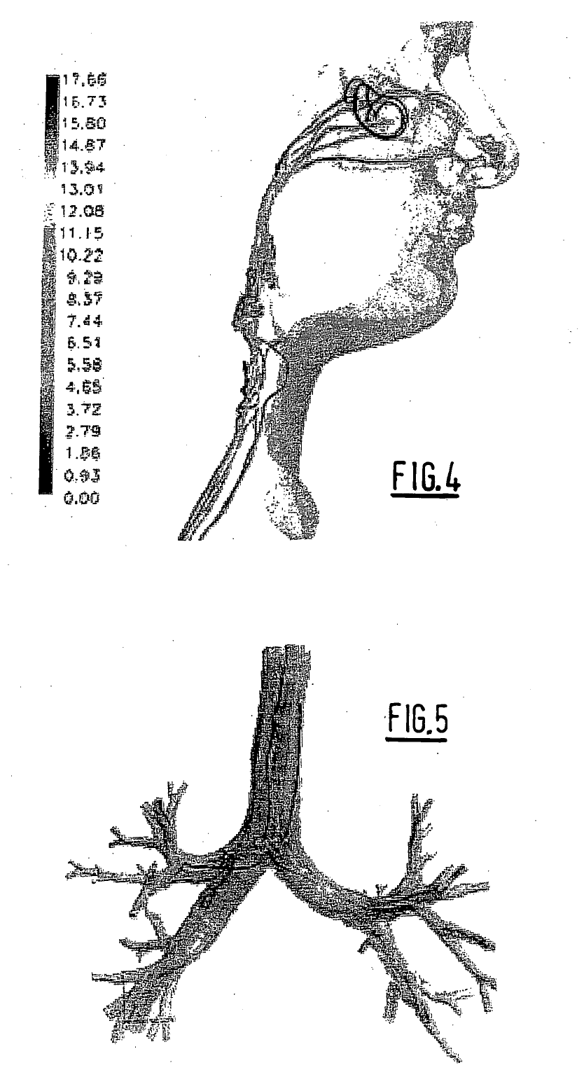

FIG. 5 represents flow patterns in the tracheobronchial tree,

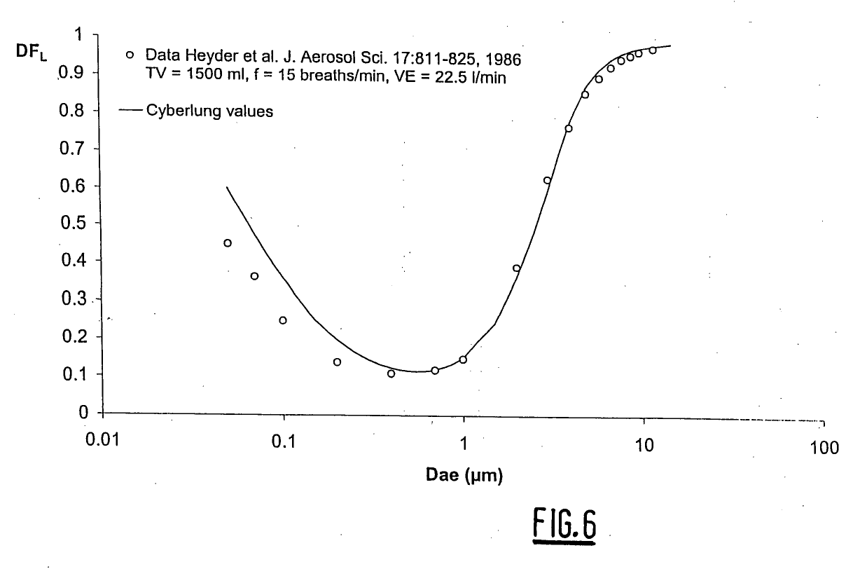

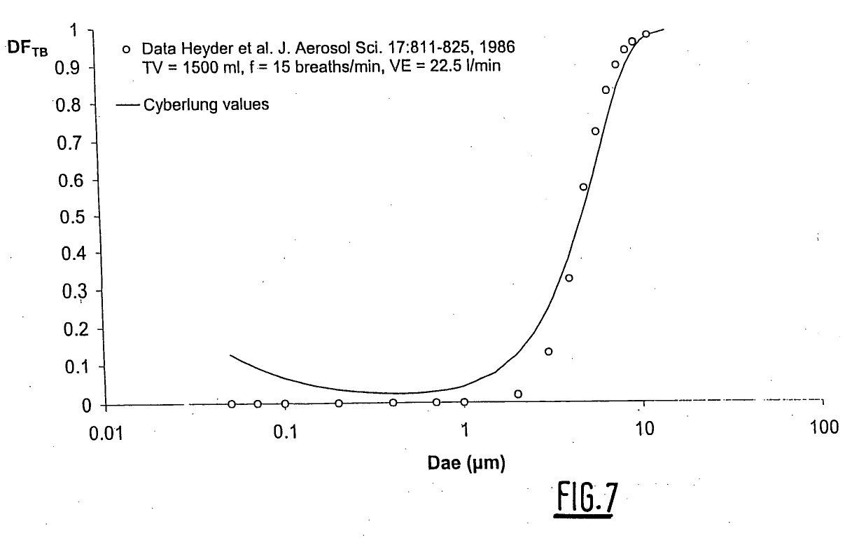

FIG. 6 represents a comparison of model predicted particle deposition patterns with data from human subject experiments for identified laboratory conditions.

FIG. 7 represents a comparison of model predicted particle deposition patterns with data from human subject experiments for identified laboratory conditions.

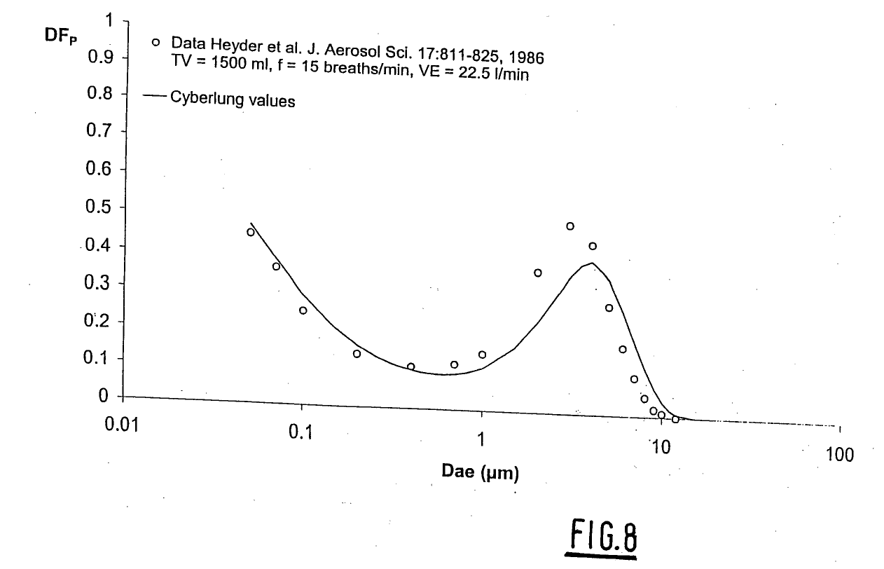

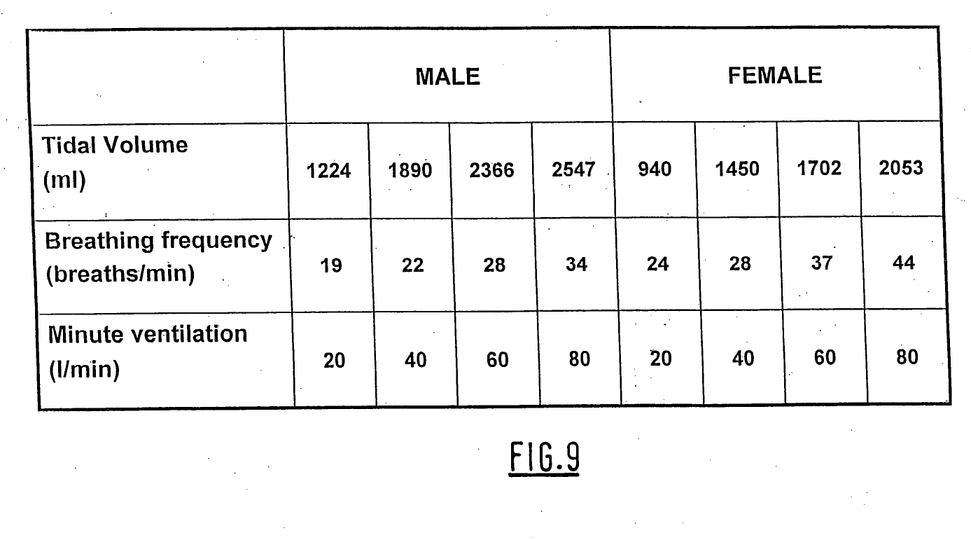

FIG. 8 represents a comparison of model predicted particle deposition patterns with data from human subject experiments for identified laboratory conditions.

FIG. 9 is a table showing the ventilatory parameters for male and female subjects,

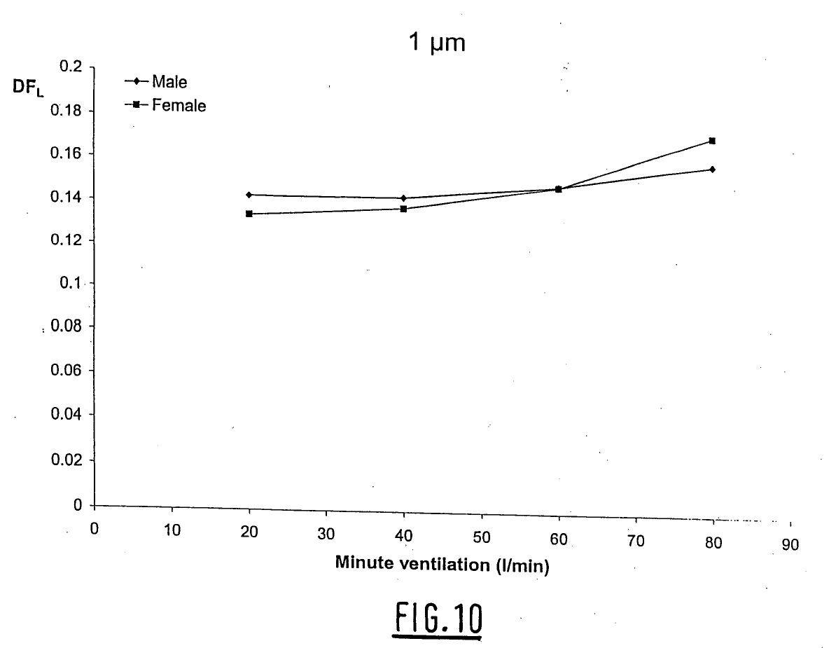

FIG. 10 shows the total lung particle deposition for male and female subjects,

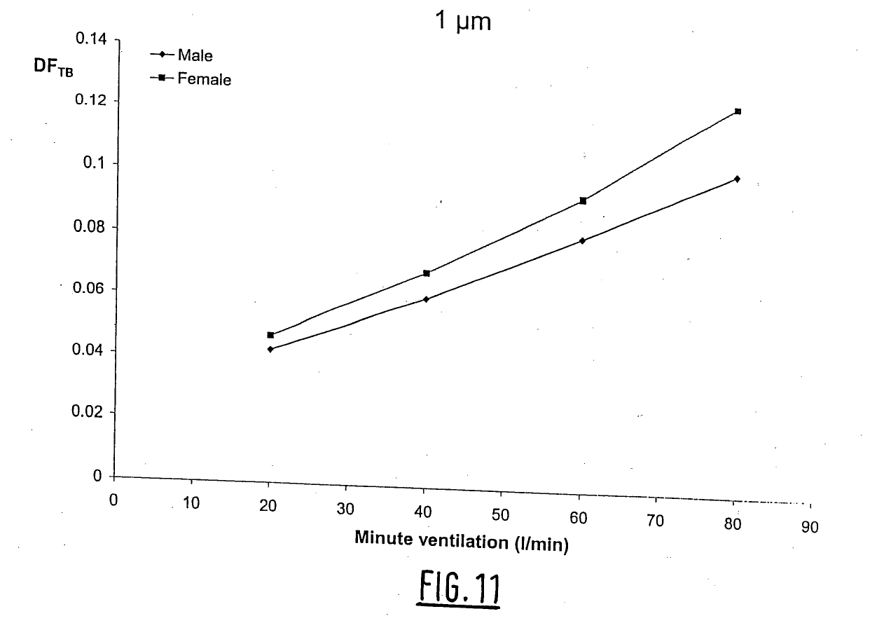

FIG. 11 shows the TB compartment deposition for male and female subjects,

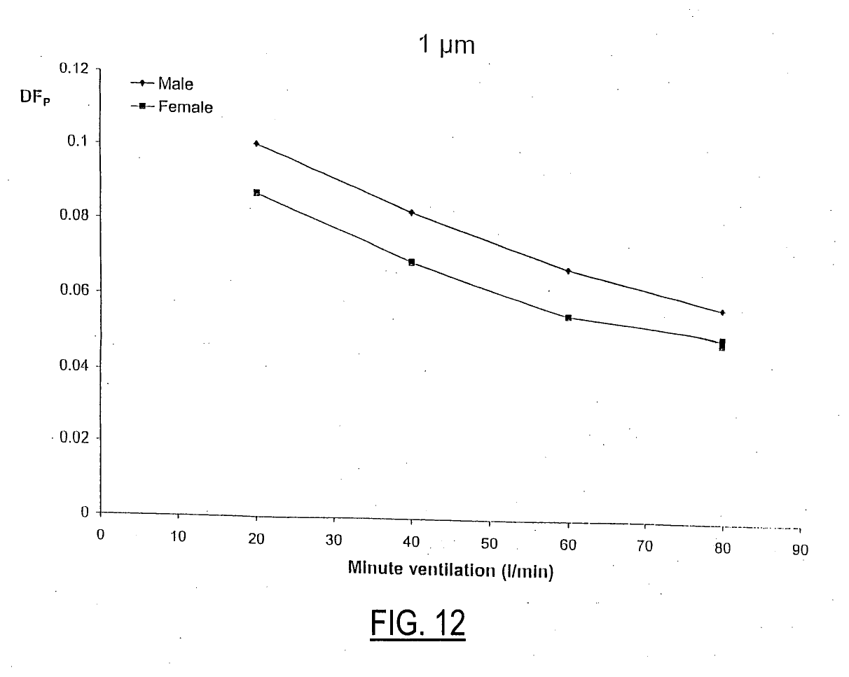

FIG. 12 shows the P compartment deposition for male and female subjects,

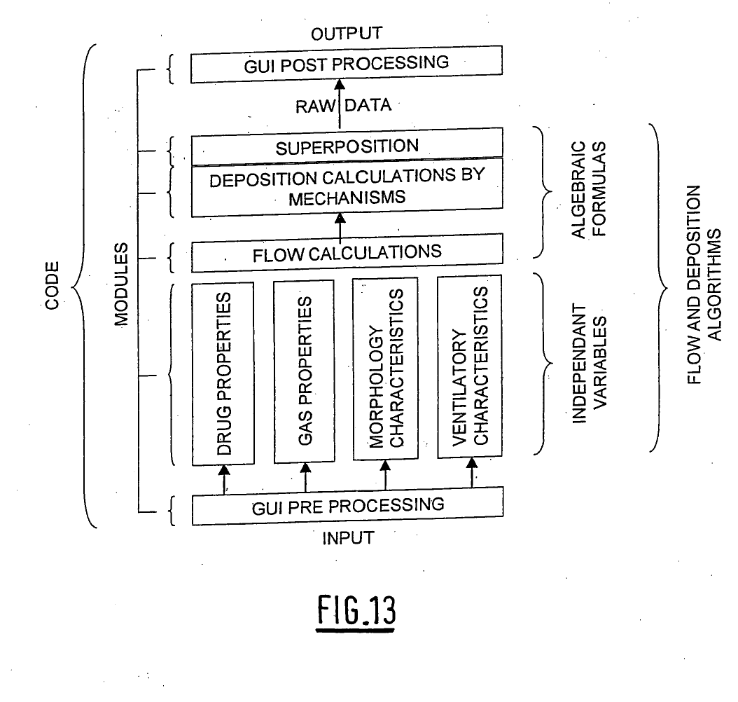

FIG. 13 shows the flowchart of the code,

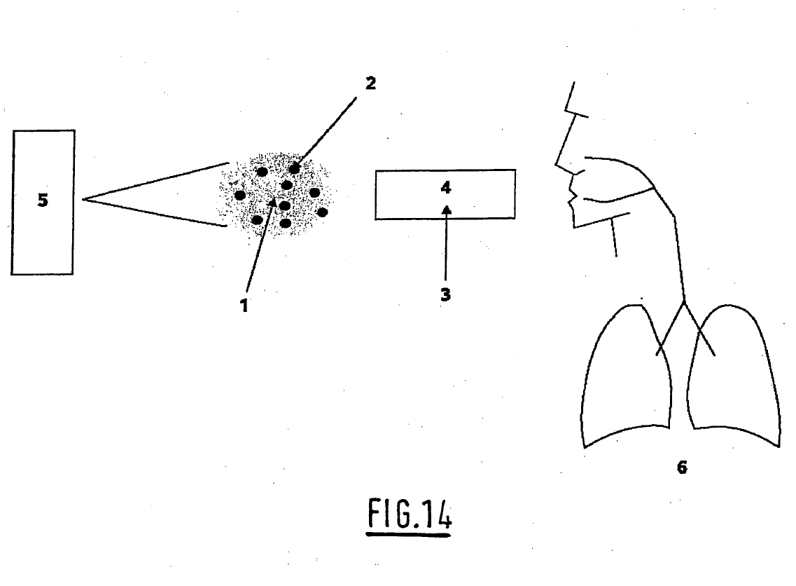

FIG. 14 represents gas as a component of an inhaled medicine, and

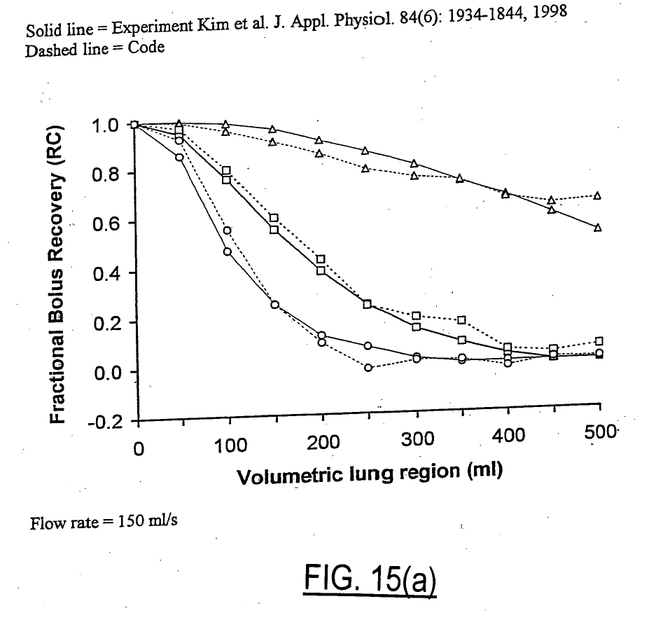

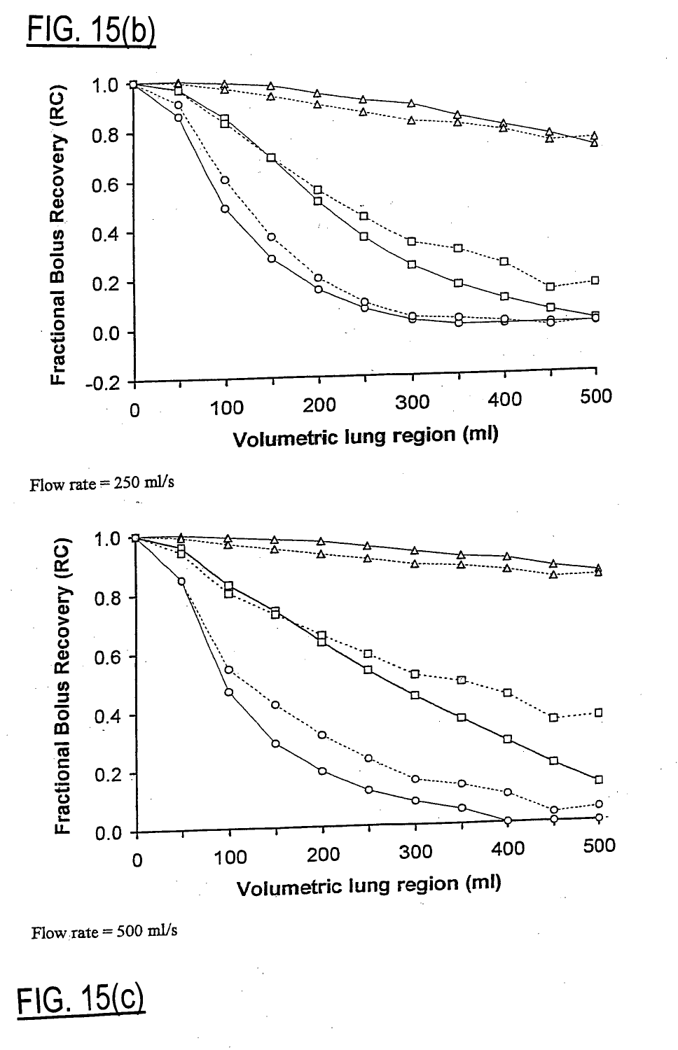

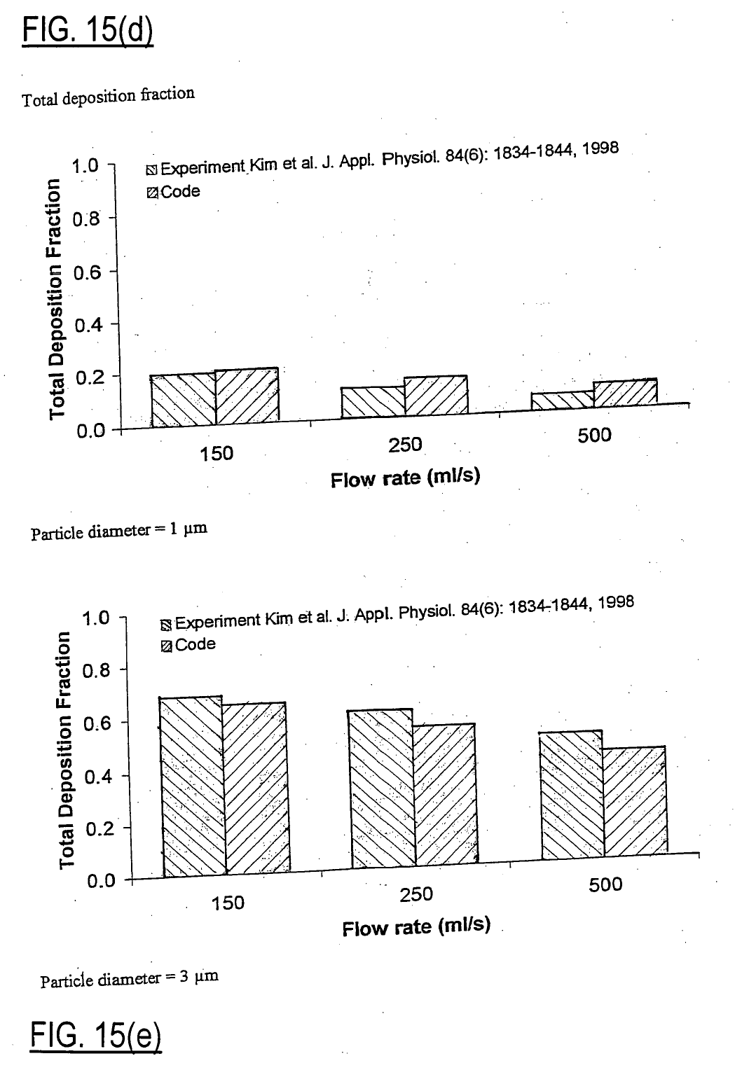

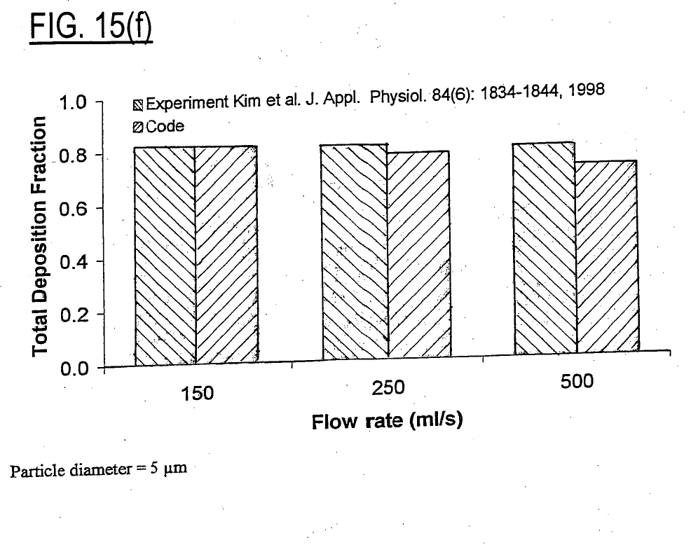

FIG. 15 is a comparison between theory and experiments in human female.

At this juncture let us summarize the situation. To solve the above identified problem, according to the present invention, a tool has been developed using a mathematical model and an associated computer code which describes the behavior and fate of inhaled gas and/or pharmacological drugs or airborne toxicants.

Said tool is a code comprising scientific/algebraic formulations and corresponding computer algorithms, which calculates flow and particle trajectories and deposition in human and animal airways as represented on the scheme of FIG. 13.

Indeed, where particles go in the respiratory system depends on the interaction of several main families of variables as described below, which are the respiratory system morphology i.e. morphology characteristics, the breathing regime i.e. ventilatory conditions, the aerosol characteristics i.e. drug and gas properties, such as air or He/O₂ mixtures.

As shown on FIG. 14, according to the present invention, an aerosol, by definition, is a multicomponent system consisting of particulate matter 2 suspended in a gaseous carrying medium 1. In other words, an aerosol has two phases: particles and gases. But, an aerosol (i.e., after generation by a medical device 5) can be mixed 4, e.g., diluted, with another gas 3. This latter gas, i.e., non-aerosol gas, can be He/O₂ mixture.

A possible way to carry out the method for designing and/or customizing the treatment or the method of treatment of a patient suffering from a respiratory or non-respiratory disease according to the present invention will be detailed below.

First, a patient suffering from said respiratory or non-respiratory disease is provided or presented to a physician or similar, so that said physician can determine the kind of disease of said patient and prescribe him/her a gas and/or a drug and a device to be used for administrating said gas and/or drug, e.g. a metered dose inhaler (MDI) or dry powder inhaler (DPI) or nebulizer.

As illustrated on FIG. 13, said physician inputs into processing means, the type of gas and/or drug prescribed and the drug particle size obtained with the prescribed device, as well as the patient characteristics and at least one morphology condition and/or ventilatory condition, e.g. tidal volume, breathing frequency and breath-hold time of said patient, as detailed below.

Then the code runs in said processing means using the input data and conditions thereby calculating gas flow and/or drug deposition characteristics customized to said patient and subsequently displaying the results.

The main families of variables that are used to calculate and display said flow and drug deposition characteristics are detailed hereafter.

Respiratory System Morphology

The input parameters regarding the morphology of the patient are a description of the lung envelope (FIG. 1), spatial orientation of the branching airway network (FIG. 2), and the dimensions of individual airways. To describe the individual airways, the input parameters are airway shape (e.g. right circular cylinders), diameters and lengths. The spatial arrangement of the individual tubes within the network are characterized by two angles: the branching angle and the gravity angle. The branching angle defines the angle between two airways, whereas the gravity angle describes their respective orientations with respect to gravity, the former being a relative measure, whereas the latter being an absolute measure.

Breathing Regime

The ventilatory input parameters are the tidal volume (TV), the breathing frequency (f) and the breathhold time (t).

The tidal volume (TV) is the amount inhaled during a breath; the breathing frequency (f) is the number of breaths per minute and the breathhold (t) is the post-inspiration time of breathholding by the patient. This assumes academic breathing pattern using constant inspiratory flow rates, constant expiratory flowrates with prescribed breath-holding times.

Aerosol Characteristics
The drug i.e. the particles:

The aerosol characteristics are the parameters of constituent particles including shapes (e.g. spherical), diameters and densities as well as the physico-chemical properties of the material. The latter properties determine the hygroscopic growth behavior of the particles within the warm humid environment of the human respiratory system.

The aforementioned parameters are for individual particles. As noted previously an innovative achievement was to treat aerosols per se.

Indeed, there is a difference between a “particle” and an “aerosol”.

An aerosol consists of a distribution of particle sizes. Therefore, for clinical applications, to address a polydisperse aerosol, the cumulative particle size distribution is divided into a number of discrete size ranges (N), each of which is treated as a monodisperse aerosol consisting of one particle size.

Therefore, to calculate the deposition pattern of a polydisperse aerosol, the code has been run N times to determine flow and deposition characteristics for each monodisperse aerosol, then appropriately weighted the respective output to determine the deposition pattern for the polydisperse aerosol.
The gas:

The gas properties of critical interest are density (ρ), absolute viscosity (μ) and mean free path (λ).

Of special interest are the He/O₂ mixtures.

The aforementioned properties affect two things, i.e. the motion of the inhaled gas per se and the trajectories of the entrained particles which are transported by said gas.

That means that different gases will have different dynamic behaviors (i.e. ρ and μ) and have different effects on entrained particles (i.e. ρ, μ and λ).

Processing: Derivation of Separate Deposition Equations for Inertial Impaction, Sedimentation and Diffusion.

The aforementioned equations are mathematically correct and biologically realistic, i.e. they portray anatomy and flow conditions and particle kinetics in vivo. The three deposition mechanisms of inhaled particles are inertial impaction, sedimentation and diffusion.

Separate equations for the aforementioned mechanisms have been defined in the literature by other authors under specific conditions corresponding to specific sites and flows. However, those conditions used in the derivations of the respective equations are inconsistent with real conditions in vivo and, hence, they should never have been coupled to simulate cumulative particle deposition processes within complete human lungs. For this reason, it would be an inappropriate act of mathematics to do so (i.e., to apply them indicrimentaly), even though if it has been done, from time to time by various authors, probably as an act of convenience.

For instance, the document of Landahl et al (Bull Math Biol; 1982)) proposes a particle deposition efficiency equation for a given condition, such as laminar flow with a parabolic velocity profile in a smooth-walled cylindrical tube of circular cross section; the document of Beeckmans (Bull Math Biol; 1982) proposes a particle deposition efficiency equation for sedimentation for the same given conditions, and the document of Ingham (Bull Math Biol; 1982) proposes a particle deposition efficiency equation for diffusion for the same given conditions.

Clearly, the theoretical conditions assumed in these documents are not compatible with in vivo situations. Hence, they cannot be applied simultaneously to different fluid motions throughout human lungs as doing it would lead to assume that different flow conditions exist simultaneously (at the same time) in a given airway, which is obviously aphysical and wrong from both biological and engineering perspectives.

Moreover, one can easily understand that applying these equations together would be inconsistent from a mathematical perspective.

In contrast, the tool that was developed in the frame of the present invention is a mathematically consistent set of deposition efficiency equations, i.e. it is based on derived equations that were legitimate. Moreover, these deposition efficiency equations have been organized into a system based on the principle of superposition.

The document Martonen, Bull Math. Biol. 1982-1983, derived separate particle deposition efficiency equations for inertial impaction, sedimentation and diffusion that are consistent with real biological features and natural flow conditions in human airways. This is an innovative achievement.

The individual equations were organized into a cohesive system describing aerosol deposition within human lungs. Importantly, novel developments integrated into the aforementioned system were the treatment of polydisperse particle size distributions an aerosol hygroscopicity.

The aforementioned components have been used to create a code that is used in the frame of the present invention.

More precisely, nine particle deposition efficiency equations as itemized in the next three sentences have been used.

First of all for turbulent flow, there should be three equations for particle deposition by inertial impaction, sedimentation and diffusion, respectively. These equations have been presented by Martonen.

Secondly, for laminar flow with a flat velocity profile there should be equations for particle deposition by inertial impaction, sedimentation and diffusion, respectively. These equations have been presented by Martonen.

Thirdly, for laminar flow with a parabolic velocity profile, there should be three equations for particle deposition by inertial impaction, sedimentation and diffusion, respectively. These equations have been presented by Landahl, Beckmans and Ingham, respectively.

Martonen (J. Pharm Sci, 1993) derived the six particle deposition efficiency equations needed for a mathematically consistent system explicitly designed for inhalation drug delivery per se, i.e. a system of equations describing particle deposition efficiency in smooth-walled tubes with no entrance effects. Indeed, it has been realized that to simulate in vivo conditions additional efforts, were needed to be biologically realistic.

Hence, significant additional advances were made in modeling which refined the code, which specific advances are defined below.

Martonen et al., Inhal Toxical. 1994; Martonen et al., Rad Prot. Dosim, 1995 incorporated natural anatomical features of cartilaginous rings in his simulations. The aforementioned reference describes the effects of rings on air motion and particle deposition. In other words, the code is based on a module that describes inherent, natural anatomical features of human airways.

Martonen et al., J Aerosol Science, 1996; Martonen et al., Aeroslo Sci Tech, 1997, described fluid dynamics entrance effects due to the inherent branching structure of the airway network in the human lungs and the influence of entrance effects on air motion and particle deposition. In other words, it is based on a module that describes inherent, natural (i.e. developing motion) flow conditions in human airways.

The subject matter of hygroscopicity is also addressed because most pharmacologic drugs are in fact hygroscopic, and in the frame of the present invention, a module is used as part of the code to simulate particle hygroscopicity and address its effects on drug deposition.

The hygroscopicity of a particle is determined by the physico-chemical properties of the constituent components, or material composition, of the particle.

Simplistically speaking, the hygroscopic characteristics of a particle describe its affinity for the uptake of water vapor. This is important because the environment of the human lung is very warm and humid. Therefore inhaled pharmacologic drugs will take up water vapor and change in size and density while traveling through lung airways.

From a mathematical point of view, the seminal problem is this: the physical dimensions and material properties of a particle are not stable but are dynamic (i.e. changing) while traveling throughout human lungs. The tool of the invention has been hence developed to permit the simulation of inhaled particles of changing properties while inside lung airways. This is important because salts e.g. NaCl are the common substrate of drug aerosols and salts are by their nature hygroscopic. Thus, the aforementioned particle deposition efficiency equations described in text above allow the physico-chemical characteristics and their commensurate effects on particle deposition to be calculated.

This is a key idea, especially for the medical usage of the tool of the invention. In other words, the tool of the invention contains a module as part of the code to describe the material properties of inhaled drugs.

The tool of the invention is flexible and can be easily modified to be able to simulate various diseased states and the effects of the physical manifestations of diseases on the gas flow and deposition characteristics of inhaled pharmacological drugs. This is accomplished in a straightforward manner. An airway disease such as asthma is described by a change in the morphology of human lungs. That is, the physical manifestation of asthma due to either bronchoconstriction or inflammation, independently or in concert, is described by a reduction in the default, i.e. control case morphology. For example, if airways in the TB are considered to be reduced by 40%, then the input airway diameters of the control case are simply multiplied by 0.6.

The advantage of this computational protocol is that a physician in the medical arena while treating an asthmatic patient can in real time run the tool of the invention on a laptop computer numerous times in a few seconds to determine the effects of diseased manifestations on the delivery of inhaled brochodilators and/or steroids.

Likewise, the effects of other diseased states, such as emphysema, cystic fibrosis, lung cancer and COPD, can be determined with the tool of the invention.

In other words, the tool of the invention integrates modules as part of the code that allow diseased states to be represented and their effects on the selective administration of inhaled gas and/or pharmacological drugs to be determined a priori.

The modules of the code have been designed based on medical clinical data. The code based calculations of gas flow and drug deposition characteristics have been compared to experimental in vitro and in vivo data for validation.

Zheng and Martonen, Cell Biochem Biophys; 1996, showed that the particle deposition patterns predicted by the code agree with in vitro data from human replica casts. Modules for biologically realistic respiratory system morphologies based on magnetic resonance imaging (MRI), computed tomography (CT) data from human subjects have been developed as part of the code, see Martonen et al., J Nucl Medicine (1998), Inhal Tox (2000), Resp Care (2000), aerosols Handbook (2005), Resp Care (2005).

The code predictions of the air flow and in vivo particle deposition have been compared with the male human subject data from experiments performed by Heyder et al. J Aerosol Sc, 1986.

Theoretical predictions and experimental measurements were in excellent agreement as shown on FIGS. 6, 7 and 8.

A module for scaling a human male morphology to a human female morphology has been developed based on parameters like height or functional residual capacity (FRC) representing the value of gas present in the respiratory system at the beginning of a breath.

The code predictions of the air flow and in vivo particle deposition have been compared with the female human subject data from experiments performed by Kym et al., J. Applied Physiology, 1998.

Theoretical predictions and experimental measurements were in excellent agreement as represented on the drawings of FIG. 15.

Computational Fluid Dynamics (CFD) and idealized flow profiles have been used to describe flow and aerosol motion throughout human lungs.

On FIG. 15, the fractional bolus recovery in women has been represented as a function of the volumetric lung region for particles of 1 (Δ), 3 (□) and 5 (o) microns in diameter with three different flow rates, using the tool of the invention, on one hand, and published data resulting from experiments, one the other hand.

The fractional bolus recovery is defined by the fraction of particles which are not deposited in the lung during the inhalation.

As one can see, the curves are roughly the same in both cases showing that the data obtained by means of the tool of the present invention are correct as they correspond to the reality.

In other words, this shows the efficiency of the tool and method of the present invention.

Output of the Tool of the Invention

The output of the tool of the invention is particle deposition patterns in various degrees of spatial resolution as represented on FIGS. 10, 11, 12, 4 and 5.

For example, absolute deposition fraction (DF) in the whole lung (DF_{L}) and the relative distribution of DF_{L} in the tracheobronchial (TB) and pulmonary (P) compartments are presented. These values are DF_{TB} and DF_{P}.

In other words DF_{TB}+DF_{P}=DF_{L}. The data are normalized to the aerosol quantity entering the trachea.

Then, the tool of the invention calculates a finer resolution among individual airway generations DF_{I}.

$$To be specific \text{:} {\sum }_{I = 0}^{16} {DF}_{I} = {DF}_{TB} and {\sum }_{I = 17}^{23} {DF}_{I} = {DF}_{P} .$$

Finally the dose delivered to each airway per unit surface area is presented.

That is the DF_{I}=(2^{I}/Surface area of a tube), where 2^{I} is the number of airways in generation I.

The tool of the invention i.e. the code is computer software based. Therefore, by definition, it also requires a hardware platform for implementation. The hardware platform can be a desktop computer or a laptop.

The software run by the tool of the invention has been purposefully written in a straightforward manner that is using simple algorithms to make it versatile. It does not require an operating system, computational capabilities or memory storage beyond current commercially available desktop or laptop computers, commonly available in the medical field.

Hence, a realistic scenario is that a physician can carry a laptop computer to implement the method or device of the present invention, e.g., on a CD-Rom, while treating patients in hospital environments. Indeed, the invention can be used by emergency service vehicles, such as ambulances, e.g., using a USB stick.

Implementing the tool of the invention is roughly based on the following steps:
a CD or USB stick comprising the software of the invention is inserted into the computer of choice, e.g. a laptop,
the computer is turned on
then, the prescribe input parameters are entered using a keyboard and a mouse,
a graphical user interface (GUI) appears on the screen the computer giving the physician options to describe the patient,
the input data are processed automatically by the installed software, i.e. the CD or USB stick
gas flow and drug deposition characteristics delivered to the patients respiratory system are computed by the tool of the invention, which takes a matter of seconds.
the physician can select using a mouse or similar, which output format is desired, e.g. total, TB, P, I, dose per unit surface area. The desired output appears electronically on the computer screen. The physician can get a hardcopy of it by using a printer which is a peripheral component of the computer system.
the physician utilizes the calculated doses to administer inhaled drugs to the patient being treated.

The information given to be physician or output are used to administer drugs via inhalation.

Regardless of the format chosen, e.g. total, TB, P, I or dose per unit surface area, the tool of the invention will present to the physician the characteristics of gas and/or drug, i.e. mass, deposited within the respiratory system.

Depending on the respiratory diseases being treated, e.g. asthma, emphysema, COPD, cancer, the issue of concern to the physician will be what degree of spatial resolution is required to treat the patient.

In some cases if the disease is spread throughout the whole lung which is the case of COPD, then obviously dose delivered to the lung will be the output of interest to the physician.

However, if the patient has asthma which is a disease of the tracheobronchial (TB) compartment, then obviously the physician will want to know dose delivered to the TB airways. But, if the patient has emphysema which is a disease of the pulmonary (P) compartments, then obviously the physician will need to know the dose delivered to the airways.

If the patient has lung cancer and the physician is using aerosol chemotherapy, then the physician will want to know deposition into an airway by airway basis the physician will want to know deposition delivered on a generation I format.

If it is a cystic fibrosis (CF) patient, then the physician will want to know dose delivered per unit surface area so receptors, i.e. localized sites, can be targeted.

### EXAMPLE

The applicability and efficiency of the present invention will be shown in the following example.

A female patient comes to the doctor with breathing problems. The physician diagnoses her using standard pulmonary tests as having asthma.

Then, to treat or design the treatment of the said patient, the doctor will use the present invention tool.

He will utilize the height and the functional residual capacity (FRC) of the patient. Actually, the FRC will already have been measured as part of the “diagnostic” series of tests.

In other words, to use the invention, the only thing the physician has to do is to measure the woman's height.

Given the woman's height and FRC, the tool of the invention generates a unique respiratory system morphology for the patient (see FIG. 3). The ventilatory condition is given by the lung function tests performed for diagnosing the said disease.

Given the kind of asthma the physician has deduced that the patient has, i.e. broncho constriction or inflammation induced, the physician will prescribe either a bronchodilatator or a steroid, or several of them as well as a gas. Said bronchodilator or steroid will be delivered by a commercially available Metered Dose Device (MDI) or a Dry Powder Inhaler (DPI) or a nebulizer that produces aerosols of a specific size. Hence, this input parameter of the tool of the invention is determined a priori.

After the physician has logged on the computer used to implement the tool of the invention, then the code runs. The code will calculate gas flow and drug deposition customized to the patient's morphology and to the output of the DPI, MDI and nebulizer a priori selected by the physician.

The tool of the invention will predict gas flow and drug deposition on a default set of ventilatory conditions to provide the physician with a control case data set. Then, the physician, based on its professional experience will decide if a different drug spatial distribution pattern would be better to treat the spatial distribution of the patient disease. If such is the case, the physician will tell to the patient how to breath. Then the physician will use the tool of the invention again to calculate gas flow and drug deposition for that new breathing regime. This iterative procedure will continue until the tool of the invention computes the spatial distribution patterns that the physician deems appropriate for the specific disease scenario or disease manifestation.

All of the above can be augmented if different gas mixtures are desired. For instance, on the original GUI, the physician can select whether the gas phase of the aerosol is air or He/O₂ mixture. Then, the tool of the invention uses the material properties of the respective gas mixtures to calculate gas flow and particle deposition.

Finally, drugs are prescribed to the patient as already described above.

In other words, as it clearly appears on FIGS. 10-12, it has been shown, using the tool of the invention, that for all particle sizes from between 0.5 to 5 μm and minute ventilations from between 20 to 80 l/min, that although the total deposition is the same for males and females there are major differences in the internal distributions of the deposited particulate matter. To be specific, the TB deposition is greater for females, whereas the pulmonary deposition is greater for males. This is of extreme importance because the TB and P compartments have clearance processes that differ in mechanisms of operation and efficiencies of operation. The TB compartment is cleansed by mucocilliary action whereas the P compartment is cleansed by macrophage action. Particles deposited in the TB compartment will be removed in about 24/34 hours but particles deposited in the P compartment may not be removed for days or weeks.

This allows to specifically address gender differences in inhalation toxicology issues and aerosol therapy regimens.

1. A method for designing and/or customizing the treatment or for treating by inhalation of a patient suffering from a respiratory or non-respiratory disease comprising the steps of:
a) presenting a patient suffering from said disease,
b) determining the kind of disease of said patient,
c) prescribing at least one drug and/or gas and at least one kind of device to be used for administrating said drug or gas, based on the determination done in step b),
d) inputting into processing means, data chosen among said drug and/or gas prescribed in step c) and drug particle size obtained with the device prescribed in step c),
e) inputting into the processing means at least one patient characteristics of the said patient,
f) inputting into processing means at least one morphology and/or ventilatory condition of said patient,
g) running a code in said processing means using the input data and conditions of step d), e) and f), thereby calculating a flow and/or deposition characteristics customized to said patient.

2. A method according to claim 1, comprising a step of targeting the pulmonary region (P) to enhance drug deposition in alveolated airways for uptake by blood for delivery to non-lung sites, in the case where the disease is a non-respiratory disease.

3. A method according to claim 1, comprising a step of targeting sites in the tracheo bronchial tree (TB) and/or in the pulmonary region (P) to enhance drug deposition in the airways, in the case where the disease is a respiratory disease.

4. A method according to claim 1, wherein the determination of the kind of disease in step b) is done by a physician.

5. A method according to claim 1, wherein, in step b), the kind of respiratory disease is chosen among asthma, emphysema, chronic obstructive pulmonary disease (COPD), cystic fibrosis and cancer.

6. A method according to claim 1, wherein, in step b), the kind of non-respiratory disease is chosen among diabetes, neurological disorders and influenza.

7. A method according to claim 1, wherein, in step c), the drug is chosen among broncho-dilatators, steroids, antibiotics, insulin, proteins, chemotherapies or gene therapies.

8. A method according to claim 1, wherein, in step c), the device is an metered dose inhaler (MDI), a dry powder inhaler (DPI), a nebulizer or a ventilator.

9. A method according to claim 1, wherein the processing means comprise a computer having at least one microprocessor for processing data, in particular for performing calculations and/or obtaining information and/or storing data and/or displaying results.

10. A method according to claim 1, wherein, the customized flow and/or deposition characteristics calculated in step g) are displayed electronically on a screen, in particular a computer monitor or similar.

11. A method according to claim 1, wherein the input of data of steps d), e) and/or f) is operated by means of a graphic user interface (GUI), and access to the GUI is accomplished via a keyboard and/or a mouse.

12. A method according to claim 1, wherein, in step g), the code comprises algorithms translating algebraic formulas describing the physics of aerosol kinetics and fluid motion in human respiratory systems for various ventilatory conditions.

13. A method according to claim 1, wherein customized flow and deposition characteristics calculated in step g) in various formats are displayed comprising total deposition in lungs, compartmental deposition within the TB tree and P region, localized deposition on a generation by generation basis, and/or mass (micrograms)-deposition on a per unit surface area basis, pressure drops on a generation by generation basis, mean velocity on a generation by generation basis.

14. A medical device for establishing and/or designing and/or customizing the treatment by inhalation of a patient suffering from a respiratory or non-respiratory disease comprising:
input/output means interactive with the processing means:
for inputting into processing means:
data chosen among a prescribed drug and/or gas and a drug particle size obtained with a prescribed inhalation device,
at least one patient characteristics and
at least one morphology and/or ventilatory condition of said patient,
and for outputting customized flow and/or deposition characteristics,
storing means for storing a code comprising a digital memory that is adapted to store algorithms translating algebraic formulas describing the physics of aerosol kinetics and fluid motion in human respiratory systems for various ventilatory conditions,
processing means cooperating with the storing means and the input means for running the code stored by the storing means and using the input data, and
displaying means for displaying customized flow and/or deposition characteristics.

15. A device according to claim 14, wherein the displaying means are a screen, in particular a computer monitor or similar.

16. A device according to claim 14, wherein the storing means for storing the code are chosen among CD-Rom, DVD-Rom, USB sticks and computer hard drives.

17. A method for designing the exposure by inhalation of a laboratory test animal used as a human surrogate in pharmacological and/or toxicological experiments, comprising the steps of:
h) presenting a selected laboratory test animal that is chosen to mimic a given human disease,
i) choosing at least one drug and/or gas to be inhaled by the test animal based on the selection done in step i),
j) choosing a device to be used for the inhalation by the test animal of the drug and/or gas chosen in step i),
k) determine operating conditions for the device chosen in step j) so that gas and/or particles of chosen sizes are produced so as to selectively deposit in the test animal;
l) inputting into processing means, data chosen among said gas and/or drug to be tested chosen in step i) and the drug particle size determined in step k),
m) inputting the morphology of the test animal,
n) inputting into processing means at least one ventilatory condition of said test animal,
o) running a code in said processing means using the input data and condition of step l), m) and n), thereby calculating a flow and/or deposition characteristics customized to said test animal.

18. A method according to claim 17, wherein the test animal is chosen among rats, mice, guinea pigs, hamsters, dogs, donkeys, cats, swine and monkeys.

19. A method for designing the exposure by inhalation of a human volunteer in toxicological experiments, comprising the steps of:
p) presenting a human volunteer,
q) choosing at least one substance to be inhaled by the human volunteer,
r) choosing a device to be used for the inhalation by the human volunteer of the substance chosen in step q),
s) determine operating conditions for the device chosen in step r) so that gas and/or particles of chosen sizes are selectively deposit in the human volunteer,
t) inputting into processing means, data chosen among said substance to be tested chosen in step t) and the substance particle size determined in step s),
u) inputting into the processing means at least one human volunteer characteristics,
v) inputting into processing means at least one morphology and/or ventilatory condition of said human volunteer,
w) running a code in said processing means using the input data and condition of step t), u) and v), thereby calculating a flow and/or deposition characteristics customized to said human volunteer.
